SEQUENCE LISTING (10 sequences)

SEQ ID NO: 1 - DNA, 48 bp, Escherichia coli
  misc_feature (35)..(36)
  misc_feature (35)..(36): n is a, c, g, or t
aaggaaatccattggcgcgccttgcgaatacgccnnygcgatgggtaa

SEQ ID NO: 2 - DNA, 48 bp, Escherichia coli
  misc_feature (35)..(36): n is a, c, g, or t
ttcctttaggtaaccgcgcggaacgcttatgcggnnrcgctacccatt

SEQ ID NO: 3 - DNA, 67 bp, Escherichia coli
  misc_feature (14)..(22): n is a, c, g, or t
aaggaaatccattnnnnnnnnnacnnnnnctccnnnnnnnttgcgaatacgccnnygcgatgggtaa

SEQ ID NO: 4 - DNA, 67 bp, Escherichia coli
  misc_feature (14)..(22): n is a, c, g, or t
ttcctttaggtaannnnnnnnntgnnnnngaggnnnnnnnaacgcttatgcggnnrcgctacccatt

SEQ ID NO: 5 - DNA, 38 bp, Escherichia coli
  misc_feature (1)..(1): n is a, c, g, or t
ntcncccttgaannnnnnnnnnnnnnnnnccccattta

SEQ ID NO: 6 - DNA, 13 bp, Escherichia coli
aaggttctaaaga

SEQ ID NO: 7 - DNA, 13 bp, Escherichia coli
aaggttataaaga

SEQ ID NO: 8 - DNA, 13 bp, Escherichia coli
gcacatttcacta

SEQ ID NO: 9 - DNA, 13 bp, Escherichia coli
ggaaggcgcacca

SEQ ID NO: 10 - DNA, 13 bp, Escherichia coli
gcacaagacacta

(19) United States
(12) Patent Application Publication
(10) Pub. No.: US 2015/0368639 A1
(43) Pub. Date: Dec. 24, 2015

(54) COMPOSITIONS, METHODS AND USES FOR MULTIPLEX PROTEIN SEQUENCE ACTIVITY RELATIONSHIP MAPPING

(71) Applicants: Ryan T. Gill, Denver, CO (US); Sean Andrew Lynch, Broomfield, CO (US); Joseph R. Warner, Boulder, CO (US)

(72) Inventors: Ryan T. Gill, Denver, CO (US); Sean Andrew Lynch, Broomfield, CO (US); Joseph R. Warner, Boulder, CO (US)

(21) Appl. No.: 14/110,072

(22) PCT Filed: Apr. 16, 2012
(86) PCT No.: PCT/US12/33799

§ 371 Date: Feb. 27, 2014

Related U.S. Application Data
(60) Provisional application No. 61/475,473, filed on Apr. 14, 2011.

(51) Int. Cl. C12N 15/10; G06F 19/22; G06F 19/28; C40B 30/02

(52) U.S. Cl. CPC C12N 15/1089; C40B 30/02; G06F 19/22; G06F 19/28

(57) ABSTRACT

Embodiments herein concern systems, compositions, methods and uses for in vivo selection of optimum target proteins of use in designing genomically-engineered cells or organisms. Some embodiments relate to compositions and methods for generating barcoded constructs of use in systems and methods described.

### CROSS-REFERENCE TO RELATED APPLICATION

This application claims priority to U.S. provisional application No. 61/475,473, filed Apr. 14, 2011, which is incorporated herein by reference in its entirety for all purposes. Apr. 14, 2012 fell on a Saturday so this application is timely filed Monday Apr. 16, 2012.

### STATEMENT REGARDING FEDERALLY SPONSORED RESEARCH OR DEVELOPMENT

This invention was made with United States government support under grant number CBET 1033397 awarded by the National Science Foundation. The United States government has certain rights in the invention.

### FIELD

Embodiments herein report compositions, systems, methods, and uses for generating comprehensive in vivo libraries related to genetic variations of target proteins. In certain embodiments, one or more proteins can be analyzed in parallel studies. In certain embodiments, one or more proteins can be prokaryotic or eukaryotic target proteins for example proteins of use in production of biofuels to biopharmaceutical agents. Some embodiments of the present invention report genetic constructs that code for one or more target protein(s) having a traceable molecular barcode outside of an open reading frame of the genetic constructs. Other embodiments include methods of generating and using these constructs. Yet other embodiments herein report systems that can include computer generated or analyzed systems having input parameters and/or methodologies for assessing and compiling certain protein mutation pools.

### BACKGROUND

Many methods for assessing genetic variation on a protein or protein function exist in the art for modifying cellular functions and for generating genetically-engineered organisms.

Microbial genomes hold the potential for tremendous combinatorial diversity, including a sequence space of about 44,600,000. Searching this diversity for genetic features that affect pertinent proteins and traits remains limited by the number of individuals that can be tested, which is a small fraction of all possibilities. Thus, strategies for first tracking all relevant genetic variations in a protein and then thoroughly evaluating them are desired. This issue has been studied in great depth at the level of individual mutations' where high-throughput methods for introducing specific mutations in residues and then mapping the effect of such mutations onto protein activity are available. Advances in genomics, and more recently multiplex DNA synthesi^{s} and homologous recombination (or recombineering) have now enabled the extension of such a strategy to the genome-scale.

### SUMMARY

Some embodiments herein report compositions, systems and methods for compiling and assessing mutational libraries of one or more target protein(s). In accordance with these embodiments, one or more target proteins can be any target protein(s). In certain embodiments, compositions, systems and methods herein can include generating mutational libraries of one or more target protein(s) wherein every change in a residue (e.g. naturally occurring or non-naturally occurring residue) of the target protein is generated and trackable. Certain embodiments, concern generating in vivo mutational libraries encompassing all possible residue changes in one or more target protein(s) to select for a trait of interest. In accordance with these embodiments, certain traits can be related to increased or decreased function (e.g. by a mutational change) and/or activity of a protein or enzyme. Systems of the present invention can include, but are not limited to, machine generated or machine analyzed systems having input parameters and/or methodologies for assessing certain genetic variations of target proteins for directed genome-engineering in cells or organisms such as microorganism, eukaryotic or prokaryotic cells.

Some embodiments herein concern constructs for compiling an in vivo trackable library of one or more target proteins (see for example FIG. 2). In accordance with these embodiments, constructs can be generated that encompass one or more genetic variation(s) of a gene or gene segment corresponding to a target protein linked to a trackable agent. In certain embodiments, the trackable agent comprises a barcode or tag. In other embodiments, the barcode is positioned outside of the open reading frame of the gene or gene segment. It is contemplated herein that genetic variations corresponding to every residue of one or more target protein(s) (e.g. proteins that make up a pathway, pharmaceutically-relevant protein etc.) can be linked to a trackable agent such as a barcode and that comprehensive in vivo libraries can be compiled using these constructs. It is contemplated that these comprehensive libraries can be generated for any eukaryotic or prokaryotic protein, trait or pathway. In certain embodiments, engineered cells or organisms can be used to produce genetically selected and/or modified target proteins identifiable by their trackable agent (e.g. barcode).

Other embodiments herein concern assessing and scoring genetic variations of genes or gene segments of one or more target proteins that affect one or more residue of the target protein(s). In accordance with these embodiments, constructs that are traced to positively affecting protein function and that contribute to an overall trait can be selected for and used for creating modulated engineered biologics, biopharma products, cells, or organisms. Certain embodiments herein provide for compiling and inputting various scores wherein the scores are linked to protein sequence-activity relationships and obtaining data related to the scores of use for a predetermined protein function or trait.

In certain embodiments, a genomically-engineered microorganism can be a eukaryotic cell, bacteria or yeast or other microorganism capable of being genomically-engineered or manipulated, for example to have improved synthesis of a byproduct of the organism. In other embodiments, compositions and methods disclosed herein to produce genomically-engineered eukaryotic or prokaryotic cells are contemplated for example, cancer cells, product-producing cells (e.g. insulin, growth factors, and other biologics), tissue cells and any others known in the art. It is contemplated that pathways capable of producing target byproducts can be optimized using embodiments disclosed herein.

In certain embodiments, scores can concern assessing protein activity changes corresponding to certain barcodes associated with specific genetic variations (e.g. residue changes, substitutions, insertions or deletions) of a target protein for example, for increased or decreased activity (e.g. enzymatic activity; protein efficacy), decreased/increased degradation or increased/decreased stability, secondary changes or tertiary changes related to folding, other physiological changes or a combination thereof.

Trackable agents contemplated of use in any of the disclosed compositions or methods can include, but are not limited to barcodes. In accordance with these embodiments, barcodes can be, but are not limited to, DNA sequences (e.g. 20-1,000 nucleotides in length) known by those skilled in the art. Since these tags are physically linked to the specific allele cassette they can be used to track the presence of each synthetic oligo as well as track each engineered cell or microorganism within a mixed population. In certain embodiments, molecular barcodes can be chosen from the experimentally verified sets used in the yeast deletion collection. In certain embodiments, barcodes can be can be further selected to exclude sequences that would lead to cleavage of DNA during library synthesis and sequences that contain more than six bases identical to the regions used to amplify the tag sequences.

In certain embodiments, performing protein sequence-activity relationship (ProSAR) mapping is described. In accordance with these embodiments, all possible residue changes in a protein can be mapped onto a phenotype conferred by that protein. It can be performed using barcodes linked specifically to each residue modification of a protein. Given the availability of tens of thousands (or more) of barcodes and the ever increasing throughput of oligo synthesis technologies (millions), this approach can be extended to allow in vivo ProSAR for dozens or more proteins simultaneously (e.g. of an entire pathway).

Some embodiments disclosed herein can include modifying microorganisms or cells to express one or more selected mutated proteins. The mutated proteins produced by the cell or microorganism can be used in any method of use for that protein.

In certain embodiments, target proteins contemplated herein can be prokaryotic or eukaryotic. In accordance with these embodiments, a target protein can be related to production of biofuels, production of a biopharmaceutical agent, enyzymatic proteins of a pathway or antibodies, fusion molecules or recombinant proteins.

### DESCRIPTION OF ILLUSTRATIVE EMBODIMENTS

### Definitions

As disclosed herein “modulate” can mean an increase, a decrease, upregulation, downregulation, an induction, a change in encoded activity, a change in stability or the like, of one or more of genes or gene clusters.

As disclosed herein “module” can mean a specific sequence of DNA designed to have a specific effect when introduced to a cell. The effect could be to target the module to a specific part of the genome or to a specific cellular location, to result in for example, a modulation as defined above, or to enable easier quantification via genomics technologies among others.

As disclosed herein “measurement of biological effect” can be a comparison of one cellular trait resulting from one genetic variation with respect to another cellular trait resulting from a second genetic variation or compared to a control with no variation. Examples of measurement of biological effect include, but are not limited to, comparison of the rate of growth of two cell types, comparison of the color of two cell types, comparison of the fluorescence of two cell types, comparison of a metabolite concentration within two cell types, comparison of lag phase of two cells types, comparison of the survival of two cell types, comparison of the consumption of a an agent by two cell types, comparison of production rates of an agent of two cell types, comparison of two or more mutations on a target protein, analysis of effects of a protein activity due to genetic variation and other parameters.

As disclosed herein “genetic modification” or “genetic variation” can mean any change(s) to a composition or structure of DNA (whole genes or gene segments) with respect to its function within an organism. Genetic modification examples include, but are not limited to, deletion of nucleotides from cell, insertion of nucleotides to cell, rearrangement of nucleotides or changes that create an amino acid change in a protein coded form by the DNA.

As disclosed herein “multiplex modification” can mean creating 2 or more genetic modifications in the same experiment. These modifications may occur within the same cell or within separate cells.

As disclosed herein “tracking module” can mean any nucleotide sequence that can be used to identify or trace a genetic modification, directly or indirectly. Tracking module examples include, but are not limited to, nucleotide sequences that can be identified by sequencing technologies, nucleotide sequences that can be identified by hybridization technologies, nucleotide sequences that create a bioproduct that can be identified, such as a protein identified by proteomic technologies or molecule identified by common analytical techniques (e.g. chromatography, spectroscopy).

As disclosed herein “functional module” can mean any nucleotide sequence inserted, rearranged, and/or removed at genetic locus (loci). A functional module elicits primary effect(s) on gene loci (locus) that can be predicted or anticipated. Functional module examples and corresponding primary effects include, but are not limited to, insertion of a promoter that cause a change of RNA transcription, alteration of nucleotides involved in translation initiation, deletion of nucleotides that make up part/all of the reading frame of a gene resulting in loss of gene product, insertion of sequence that causes a change in gene product, and deletion of sequence that interacts with a small molecule that causes an effect to be less dependent on the small molecule.

### BRIEF DESCRIPTION OF THE FIGURES

The following drawings form part of the present specification and are included to further demonstrate certain embodiments of the present invention. The embodiments may be better understood by reference to one or more of these drawings in combination with the detailed description of specific embodiments presented herein.

FIGS. 1A-1B represent generating a construct of use for certain embodiments disclosed herein.

FIG. 2 represents an exemplary method for generating certain constructs of some embodiments disclosed herein.

FIGS. 3A-3B represent an exemplary cloning method for a target gene comprising a selectable marker in linear and circularized form.

FIG. 4 represents an exemplary method for amplifying constructs of certain embodiment described herein.

FIG. 5 represents an exemplary method for generating single stranded DNA including various markers described in certain embodiments.

FIG. 6 represents an exemplary construct of some embodiments reported herein.

FIGS. 7A-7B illustrate (A) a schematic of eukaryotic protein sequence-activity relationship (ProSAR) mapping and (B) a construct.

FIG. 8 illustrates an exemplary strategy for multiplex recombineering ProSAR.

FIGS. 9A and 9B illustrates (a) an exemplary design of the synthetic oligonucleotide and (b) an oligo amplification process from design to recovery. Recovered oligos will be used in the next steps of library creation.

FIG. 10 represents a schematic of steps in library construction between oligo recovery and double-stranded recombination.

FIGS. 11A and 11B represent a schematic of library construction using single-stranded oligonucleotides. (a) General oligo design (ex. FIG. 9b) and (b) Oligo recovery and recombineering for library generation.

FIGS. 12A-12B represent electrophoretic separation of constructs disclosed herein: (a) Assymetric PCR with five oligos in multiplex and (b) Colony PCR on a small sample of transformants after barcode-swapping.

### DETAILED DESCRIPTION

In the following sections, various exemplary compositions and methods are described in order to detail various embodiments of the invention. It will be obvious to one skilled in the art that practicing the various embodiments does not require the employment of all or even some of the details outlined herein, but rather that concentrations, times, temperature and other details may be modified through routine experimentation. In some cases, well known or previously disclosed methods or components have not been included in the description.

In accordance with embodiments of the present invention, there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition 1989, Cold Spring Harbor Laboratory Press, Cold Spring Harbor, N.Y.; Animal Cell Culture, R. I. Freshney, ed., 1986).

### Combinatorial Selections and Small Gene Pool Selection Technologies

In some embodiments, methods described herein include identifying genetic variations of one or more target gene that affect one or more, or all residues of one or more target proteins. In accordance with these embodiments, compositions and methods disclosed herein permit parallel analysis of two or more target proteins or proteins that contribute to a trait. Parallel analysis of multiple proteins by a single experiment described can facilitate identification, modification and design of superior systems for example for producing a eukaryotic or prokaryotic by product, producing a eukaryotic byproduct (e.g. biological agent such as a growth factor, antibody etc) in a prokaryotic organism and the like. Relevant biologics used in analysis and treatment of disease can be produced in these genetically engineered environments that could reduce production time, increase quality all while reducing costs to the manufacturers and the consumers.

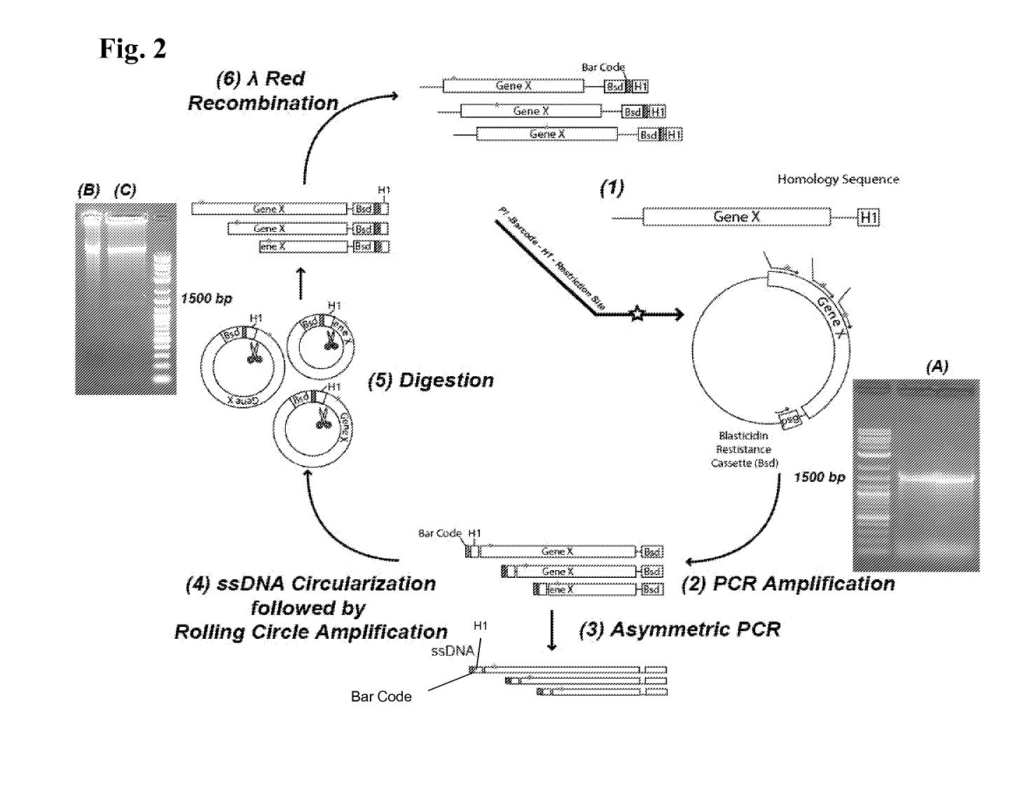

Other embodiments disclosed herein report constructs of use for studying genetic variations of a gene or gene segment wherein the gene or gene segment is capable of generating a protein. In accordance with these embodiments, a construct can be generated for one, two or all residue modifications of a target protein that is linked to a trackable agent (e.g. a barcode). In certain embodiments, a barcode indicative of a genetic variation of a gene of a target protein can be located outside of the open reading frame of the gene (see for example, FIG. 2 of the Example section). It is contemplated herein that these methods can be performed in vivo. Constructs described herein can be used to compile a comprehensive library of genetic variations encompassing all residue changes of one target protein, more than one target protein or target proteins that contribute to a trait. In certain embodiments, libraries disclosed herein can be used to select proteins with improved qualities to create an improved single or multiple protein system for example for producing a byproduct (e.g. chemical, biofuels, biological agent, pharmaceutical agent, for biomass etc) or biologic compared to a non-selective system.

### Protein Sequence-Activity Relationship (ProSAR) Mapping

Understanding the relationship between a protein's amino acid structure and its overall function continues to be of great practical, clinical and scientific significance for biologists and engineers. Directed evolution can be a powerful engineering and discovery tool, but the random and often combinatorial nature of mutations makes their individual impacts difficult to quantify and thus challenges further engineering. More systematic analysis of contributions of individual residues (e.g., saturation mutagenesis) remains labor- and time-intensive for entire proteins and simply is not possible on reasonable timescales for multiple proteins in parallel (metabolic pathways, multi-protein complexes) using standard methods.

Advances in multiplex oligonucleotide synthesis, recombineering, and DNA assembly are radically changing genetic engineering with broad implications across biology and biotechnology in general. This technology can be used to rapidly and efficiently examine the roles of all genes in a microbial or eukaryotic genome using mixtures of barcoded oligonucleotides. Here, these compositions and methods can be used develop a powerful new technology for comprehensively mapping protein structure-activity relationships (ProSAR).

As disclosed herein, certain embodiments combine multiplex oligonucleotide synthesis with recombineering, to create libraries of specifically designed and barcoded mutations along a gene of interest in parallel and on laboratory time scales. Screens and/or selections followed by high-throughput sequencing and/or barcode microarray methods then allow for rapid mapping of protein sequence-activity relationships (PROSAR). The central hypothesis is that systematic PROSAR mapping can elucidate individual amino acid mutations for improved function and/or activity and/or stability etc. The process can then be iterated to combinatorially improve the function, activity or stability. Given existing capabilities of multiplex oligo synthesis (about 120,000+ oligos/array) and recombineering, it will be possible to scale this approach to construct libraries for dozens (e.g., complete substitutions) to hundreds (e.g., alanine scanning etc.) of proteins in a single experiment.

Understanding the relationships between a protein's amino acid structure and function is critical in protein engineering efforts, which are increasingly commonplace in almost all drug development programs (e.g. whether focused on protein-based therapies or enzyme driven synthesis of pharmaceutical products). Now, protein design criteria grow increasingly stringent, including efforts to simultaneously alter multiple characteristics such as overall stability, catalytic activity, pharmacokinetic activity, shelf life, among others depending on the application.

While many powerful methods for engineering protein function have been reported, all of such efforts have been fundamentally limited by available throughput in DNA synthesis, construction, and sequencing technologies. DNA sequencing technology has advanced to the point that sequencing of full length genes (and many variants) became accessible to many research laboratories, enabling an explosion in methods for directed protein evolution, rational protein engineering, sequence-to-activity mapping, and combinations thereof. Then, DNA synthesis technologies underwent a similar step change in throughput, where it is now possible to synthesize sufficient DNA to cover the E. coli genome several times over on a single DNA microarray. Therefore, rational protein libraries are now possible. Strategies to construct barcoded, complete substitution libraries for several different proteins at the same time and for dramatically reduced costs per protein are described herein. Using existing multiplex DNA synthesis technology, as disclosed, a complete substitution library for a protein construct can be barcoded (or non-barcoded, if desired) for several hundred proteins at the same time.

Embodiments herein apply to analysis and structure/function/stability library construction of any protein with a corresponding screen or selection for activity. Library size depends on the number (N) of amino acids in a protein of interest, with a full saturation library (all 20 amino acids at each position or non-naturally—occurring amino acids) scaling as 19 (or more)×N and an alanine-mapping library scaling as 1×N. Thus, screening of even very large proteins of more than 1,000 amino acids is tractable given currently multiplex oligo synthesis capabilities (e.g. 120,000 oligos). In addition to activity screens, more general properties with developed high-throughput screens and selections could be efficiently tested using our libraries. For example, universal protein folding and solubility reporters have been engineered for expression in the cytoplasm, periplasm, and the inner membrane. Moreover, due to the designed single nature of mutations (e.g., no background mutations) screening of the same protein library under different conditions (e.g., different temperatures, different substrates or co-factors, etc.) permits identification of residue changes required for expression of various traits (design criteria). In other embodiments, because residues are analyzed one at a time, mutations at residues important for a particular trait (e.g., thermostability, resistant to environmental pressures, increased or decrease in functionality or production) could be combined via multiplex recombineering with mutations important for various other traits (e.g. catalytic activity) to create combinatorial libraries for multi-trait optimization.

In certain embodiments, methods for creating and/or evaluating comprehensive, in vivo, mutational libraries of one or more target protein(s) has been described. This approach can be extended via a barcoding technology to generate trackable mutational libraries for every residue in a protein. This approach can be based on protein sequence-activity relationship mapping method extended to work in vivo, capable of working on a few to hundreds of proteins simultaneously depending on the technology selected. These methods permit one to map in a single experiment all possible residue changes over a collection of desired proteins onto a trait of interest, as part of individual proteins of interest or as part of a pathway. This approach can be used at least for the following by mapping i) all residue changes for all proteins in a specific biochemical pathway (e.g. lycopene production) or that catalyze similar reactions (e.g. dehydrogenases or other enzymes of a pathway of use to produce a desired effect or produce a product) or ii) all residues in the regulatory sites of all proteins with a specific regulon (e.g. heat shock response) or iii) all residues of a biological agent used to treat a health condition (e.g. insulin, a growth factor (HCG), an anti-cancer biologic, a replacement protein for a deficient population etc).

Certain embodiments concern assigning scores related to various input parameters in order to generate one or more composite score(s) for designing genomically-engineered organisms or systems. These scores can reflect quality of genetic variations in genes or genetic loci as they relate to selection of an organism or design of an organism for a predetermined production, trait or traits. Certain organisms or systems may be designed based a need for improved organisms for biorefining, biomass (crops, trees, grasses, crop residues, forest residues, etc), biofuel production and using biological conversion, fermentation, chemical conversion and catalysis to generate and use compounds, biopharmaceutical production and biologic production. In certain embodiments, this can be accomplished by modulating growth or production of microorganism through genetic manipulation disclosed herein.

Genetic manipulation (e.g. using genes or gene fragments disclosed herein) of genes encoding a protein can be used to make desired genetic changes that can result in desired phenotypes and can be accomplished through numerous techniques including but not limited to, i) introduction of new genetic material, ii) genetic insertion, disruption or removal of existing genetic material, as well as, iii) mutation of genetic material (e.g. point mutations) or any combinations of i,ii, and iii, that results in desired genetic changes with desired phenotypic changes. Mutations can be directed (e.g. site-directed) or random, utilizing any techniques such as insertions, disruptions or removals, in addition to those including, but not limited to, error prone or directed mutagenesis through PCR, mutator strains, and random mutagenesis.

In protein engineering, it is desired to study combinations of genetic variations (e.g. point mutations) that improve the activity, stability or reduced cross reactivity of a particular protein in vivo. A multiplex recombineering approach to enable such studies at a scale and resolution not previously possible is presented herein. While the previously described methods focus on ribosome binding site modulation, a new approach where collections of oligonucleotides are designed to either modify residues within i) a specific protein of interest or ii) across a set of proteins of collective interest (see for example FIG. 2, a pathway) are described. While many methods for directed protein evolution exist, embodiments presented herein have increased utility because it can be employed in vivo and in a highly parallel fashion across a group of proteins.

The global transcription machinery has been targeted as a means to engineer global changes in gene expression for bacteria and yeast in the laboratory. Such a method can have the following advantages: i) no in vitro cloning is needed, ii) sequence diversity is directed towards known DNA binding regions, therefore there is a higher probability of finding improved sequences with a smaller library size, and iii) several transcription factors may be engineered in multiplex due to the smaller library size.

In some embodiments herein, disclosed methods demonstrate abilities for inserting and accumulating higher order modifications into a microorganism's genome or a target protein; for example, multiple different site-specified mutations in the same genome, at high efficiency to generate libraries of genomes with over 300 targeted modifications are described. These mutations are not confined only to sequences of regulatory modules, but can also extend to protein-coding regions. Protein coding modifications can include, but are not limited to, amino acid changes, codon optimization, and translation tuning

### Nucleic Acids

In various embodiments, isolated nucleic acids may be introduced to a microorganism to modulate growth of the microorganism, for example, to increase tolerance to a toxic chemical. The isolated nucleic acid may be derived from genomic RNA or complementary DNA (cDNA). In other embodiments, isolated nucleic acids, such as chemically or enzymatically synthesized DNA, may be of use for capture probes, primers and/or labeled detection oligonucleotides.

A “nucleic acid” can include single-stranded and/or double-stranded molecules, as well as DNA, RNA, chemically modified nucleic acids and nucleic acid analogs. It is contemplated that a nucleic acid may be of 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, about 110, about 120, about 130, about 140, about 150, about 160, about 170, about 180, about 190, about 200, about 210, about 220, about 230, about 240, about 250, about 275, about 300, about 325, about 350, about 375, about 400, about 425, about 450, about 475, about 500, about 525, about 550, about 575, about 600, about 625, about 650, about 675, about 700, about 725, about 750, about 775, about 800, about 825, about 850, about 875, about 900, about 925, about 950, about 975, about 1000, about 1100, about 1200, about 1300, about 1400, about 1500, about 1750, about 2000 or greater nucleotide residues in length, up to a full length protein encoding or regulatory genetic element.

### Construction of Nucleic Acids

Isolated nucleic acids may be made by any method known in the art, for example using standard recombinant methods, synthetic techniques, or combinations thereof. In some embodiments, the nucleic acids may be cloned, amplified, or otherwise constructed.

The nucleic acids may conveniently comprise sequences in addition to a portion of a lysine riboswitch. For example, a multi-cloning site comprising one or more endonuclease restriction sites may be added. A nucleic acid may be attached to a vector, adapter, or linker for cloning of a nucleic acid. Additional sequences may be added to such cloning and sequences to optimize their function, to aid in isolation of the nucleic acid, or to improve the introduction of the nucleic acid into a cell. Use of cloning vectors, expression vectors, adapters, and linkers is well known in the art.

### Recombinant Methods for Constructing Nucleic Acids

Isolated nucleic acids may be obtained from bacterial or other sources using any number of cloning methodologies known in the art. In some embodiments, oligonucleotide probes which selectively hybridize, under stringent conditions, to the nucleic acids of a bacterial organism. Methods for construction of nucleic acid libraries are known and any such known methods may be used.

### Nucleic Acid Screening and Isolation

Bacterial RNA or cDNA may be screened for the presence of an identified genetic element of interest using a probe based upon one or more sequences. Various degrees of stringency of hybridization may be employed in the assay.

High stringency conditions for nucleic acid hybridization are well known in the art. For example, conditions may comprise low salt and/or high temperature conditions, such as provided by about 0.02 M to about 0.15 M NaCl at temperatures of about 50° C. to about 70° C. Other exemplary conditions are disclosed in the following Examples. It is understood that the temperature and ionic strength of a desired stringency are determined in part by the length of the particular nucleic acid(s), the length and nucleotide content of the target sequence(s), the charge composition of the nucleic acid(s), and by the presence or concentration of formamide, tetramethylammonium chloride or other solvent(s) in a hybridization mixture. Nucleic acids may be completely complementary to a target sequence or may exhibit one or more mismatches.

### Nucleic Acid Amplification

Nucleic acids of interest may also be amplified using a variety of known amplification techniques. For instance, polymerase chain reaction (PCR) technology may be used to amplify target sequences directly from bacterial RNA or cDNA. PCR and other in vitro amplification methods may also be useful, for example, to clone nucleic acid sequences, to make nucleic acids to use as probes for detecting the presence of a target nucleic acid in samples, for nucleic acid sequencing, or for other purposes.

### Synthetic Methods for Constructing Nucleic Acids

Isolated nucleic acids may be prepared by direct chemical synthesis by methods such as the phosphotriester method, or using an automated synthesizer. Chemical synthesis generally produces a single stranded oligonucleotide. This may be converted into double stranded DNA by hybridization with a complementary sequence or by polymerization with a DNA polymerase using the single strand as a template. While chemical synthesis of DNA is best employed for sequences of about 100 bases or less, longer sequences may be obtained by the ligation of shorter sequences.

### Protein Methodologies

Any method known in the art for identifying, isolating, purifying, using and assaying activities of target proteins contemplated herein are contemplated. Target proteins contemplated herein include protein agents used to treat a human condition or to regulate processes (e.g. part of a pathway such as an enzyme) involved in disease of a human or non-human mammal. Any method known for selection and production of antibodies or antibody fragments is also contemplated.

### Computer Programs

Embodiments of the present invention may be provided as a computer program product which may include a machine-readable medium having stored thereon instructions which may be used to program a computer (or other electronic devices) to perform a process. The machine-readable medium may include, but is not limited to, floppy diskettes, optical disks, compact disc read-only memories (CD-ROMs), and magneto-optical disks, ROMs, random access memories (RAMs), erasable programmable read-only memories (EPROMs), electrically erasable programmable read-only memories (EEPROMs), magnetic or optical cards, flash memory, or other type of media/machine-readable medium suitable for storing electronic instructions. Moreover, embodiments of the present invention may also be downloaded as a computer program product, wherein the program may be transferred from a remote computer to a requesting computer by way of data signals embodied in a carrier wave or other propagation medium via a communication link (e.g., a modem or network connection).

For the sake of illustration, various embodiments of the present invention have herein been described in the context of computer programs, physical components, and logical interactions within modern computer networks. While these embodiments describe various aspects in relation to modern computer networks and programs, methods and apparati described herein are equally applicable to other systems, devices, and networks as one skilled in the art will appreciate. As such, the illustrated applications of the embodiments are not meant to be limiting, but instead exemplary. In addition, embodiments are applicable to all levels of computing from the personal computer to large network mainframes and servers.

The term “component” refers broadly to a software, hardware, or firmware (or any combination thereof) component. Components are typically functional components that can generate useful data or other output using specified input(s). A component may or may not be self-contained. An application program (also called an “application”) may include one or more components, or a component can include one or more application programs.

Some embodiments include some, all, or none of the components along with other modules or application components. Still yet, various embodiments may incorporate two or more of these components into a single module and/or associate a portion of the functionality of one or more of these components with a different component.

The term “memory” can be any device or mechanism used for storing information. In accordance with some embodiments of the present invention, memory is intended to encompass any type of, but is not limited to, volatile memory, nonvolatile memory and dynamic memory. For example, memory can be random access memory, memory storage devices, optical memory devices, magnetic media, floppy disks, magnetic tapes, hard drives, SIMMs, SDRAM, DIMMs, RDRAM, DDR RAM, SODIMMS, erasable programmable read-only memories (EPROMs), electrically erasable programmable read-only memories (EEPROMs), compact disks, DVDs, and/or the like. In accordance with some embodiments, memory may include one or more disk drives, flash drives, databases, local cache memories, processor cache memories, relational databases, flat databases, and/or the like. In addition, those of ordinary skill in the art will appreciate many additional devices and techniques for storing information can be used as memory.

Memory may be used to store instructions for running one or more applications or modules on processor. For example, memory could be used in some embodiments to house all or some of the instructions needed to execute the functionality of one or more of the modules and/or applications illustrated in FIG. 2.

### Exemplary Computer System Overview

Embodiments herein can include various steps. A variety of these steps may be performed by hardware components or may be embodied in machine-executable instructions, which may be used to cause a general-purpose or special-purpose processor programmed with the instructions to perform the steps. Alternatively, the steps may be performed by a combination of hardware, software, and/or firmware.

The components described above are meant to exemplify some types of possibilities. In no way should the aforementioned examples limit the scope of the invention, as they are only exemplary embodiments.

### EXAMPLES

The following examples are included to illustrate various embodiments. It should be appreciated by those of skill in the art that the techniques disclosed in the examples that follow represent techniques discovered to function well in the practice of the claimed methods, compositions and apparatus. However, those of skill in the art should, in light of the present disclosure, appreciate that many changes may be made in the embodiments which are disclosed and still obtain a like or similar result without departing from the spirit and scope of the invention.

### Example 1

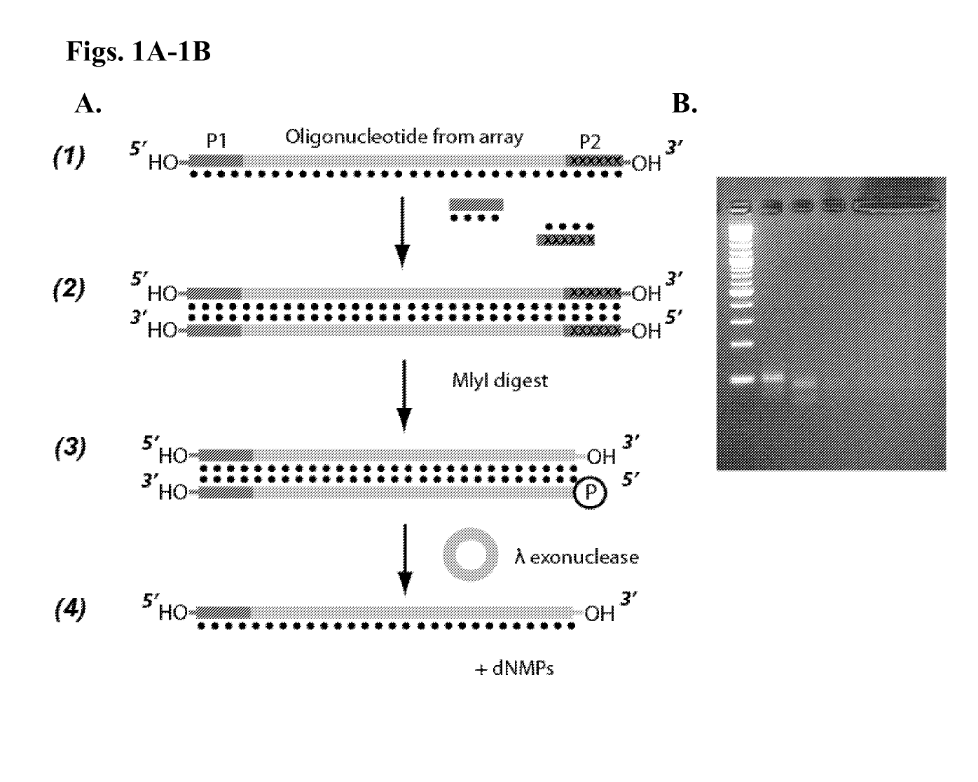

FIG. 1A represents a generalized method for the amplification of a stock of single-stranded DNA oligonucleotides obtained via parallel DNA synthesis and the subsequent regeneration for use in a PCR reaction. Any method known in the art may be used for amplification. In one exemplary method, a plan for amplifying a set of oligos synthesized in parallel was devised (ssDNA to dsDNA) thus creating a stock of DNA and subsequently regenerate ssDNA (dsDNA to ssDNA) with which to employ in future PCR reactions. This has been accomplished with a single oligonucleotide using the following protocol and can be extended to amplify a stock of oligos obtained via parallel DNA synthesis.

1. A ssDNA oligo (e.g. a 100-mer) was obtained containing the necessary homology sequence and mutation flanked by priming sites P1 and P2. Priming site P2 is unique in that it contains a restriction site (e.g. MlyI). MlyI is an example of a TypeIIS endonuclease that cleaves DNA 5 bp away from its recognition sequence. In a multiplex context, these priming sites would be present in all synthesized DNA molecules.

2. An amplification reaction (PCR) is carried out using short primers designed to amplify the ssDNA from priming sites P1 and P2 to yield dsDNA.

3. This dsDNA can then be digested with MlyI to remove P2 sequence and generate a blunt end with 5′ end of the complementary strand being phosphorylated. Since the sense strand of DNA was amplified using forward primer P1, it will remain without a phosphate.

4. The digested DNA is then subjected to lambda exonuclease digestion. Lambda exonuclease degrades duplex DNA in a 5′ to 3′ direction. Since a 5′ phosphate is required for initiation, only the complementary strand is degraded leaving a ssDNA molecule that can be used for PCR.

FIG. 1B illustrates a gel where the far left lane is base pair (bp) ladder; Lane 1: PCR amplification of 100 mer oligo; Lane 2: MlyI digest removing ˜20 bp; Lane 3 and 4: Lambda exo digest. Faint intensity due to the decreased amount of EtBr that can intercalate with ssDNA.

### Example 2

FIG. 2 represents a generalized method for introducing barcoded point mutations throughout a gene or pathway using for example, recombineering. This application is applicable to prokaryotic and eukaryotic genes.

1. Representation of hypothetical Gene X on the chromosome flanked downstream by homology region (H1). The desired gene is cloned into a plasmid upstream of a antibiotic resistance marker (e.g. blasticidin resistance marker, bsd). Oligonucleotides are designed and synthesized in parallel such that the following features are present (5′-3′): priming site P1, a molecular barcode, homology region H1, a unique Type II S restriction site, a sequence annealing to the template containing a specific point mutation and priming site P2 which contains a restriction site (e.g. MlyI). Oligonuleotides are then amplified to create dsDNA using priming sites P1 and P2. Amplified dsDNA is digested with MlyI to remove priming site P2 and subsequently digested with lambda exonuclease to generate ssDNA (See FIG. 2, (1)).

2. Using the plasmid DNA from (1), ssDNA oligonucleotides are employed in PCR reactions with a common downstream primer to amplify dsDNA containing a specific barcoded mutation (see the representative photo of an electrophoresis gel, lane A, on figure with DNA at ˜1500 bp).

3. Amplified dsDNA is then used as a template in an asymmetric amplification reaction (e.g. PCR) using 1:1000 ratio of forward to reverse primer. Reverse primer is phosphorylated for subsequent circularization using CircLigase from Epicentre. *Note* asymmetric PCR reaction can be optional because a linear dsDNA molecule can be circularized using T4 DNA ligase. However, according to the manufacturer, circularization of ssDNA using CircLigase yields little to none of the concatamers that can potentially form when circularizing dsDNA.

4. DNA polymerase (e.g Phi29) is then used in rolling circle amplification (RCA) of circularized ssDNA (or dsDNA) using random hexamer primers (see the representative photo of an electrophoresis gel, lane B).

5. RCA reaction is then precipitated using butanol and subsequently digested with unique Type IIS restriction enzyme to yield dsDNA of the original length with barcode removed from coding region (see the representative photo of an electrophoresis gel, lane C).

6. Digested DNA is gel extracted and subsequently used for recombineering to generate gene X with a point mutation and a corresponding barcode. It is contemplated herein that for any protein all residues of the protein can be mutated (tracked by a specific barcode) and assessed for biological function/contribution.

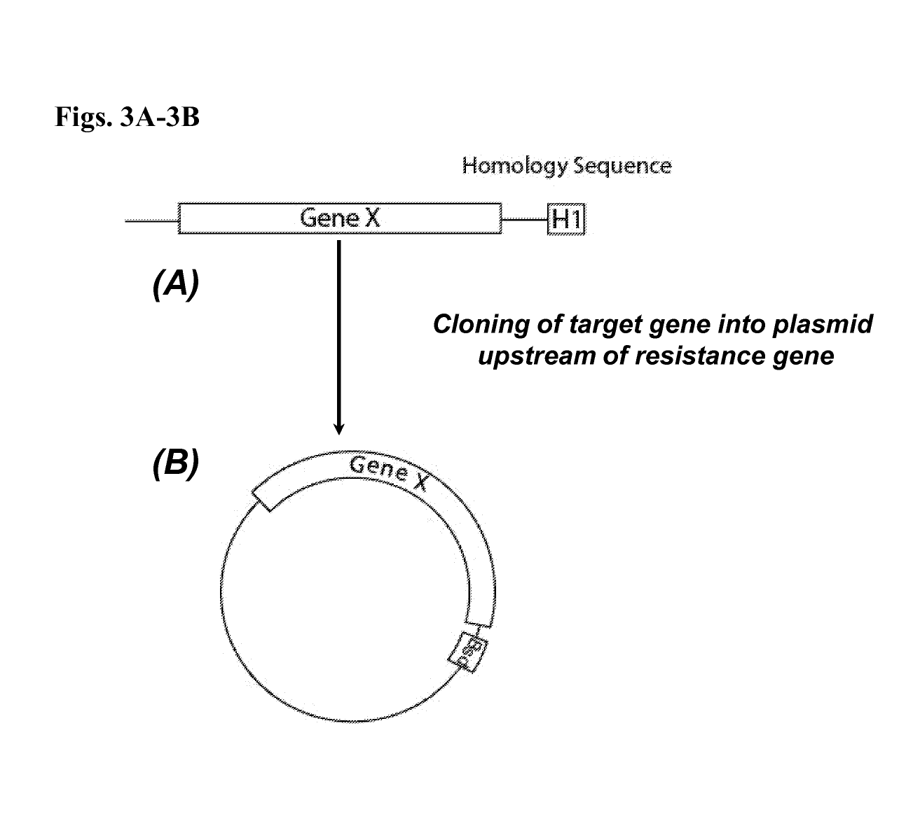

FIGS. 3 to 5 represent an expanded version of FIG. 2. FIG. 3 represents FIG. 2(1) 1. (A) Representation of hypothetical Gene X on the chromosome flanked downstream by homology region H1. (B) The desired gene is cloned into a plasmid upstream of an antibiotic resistance marker (e.g. blasticidin, bsd).

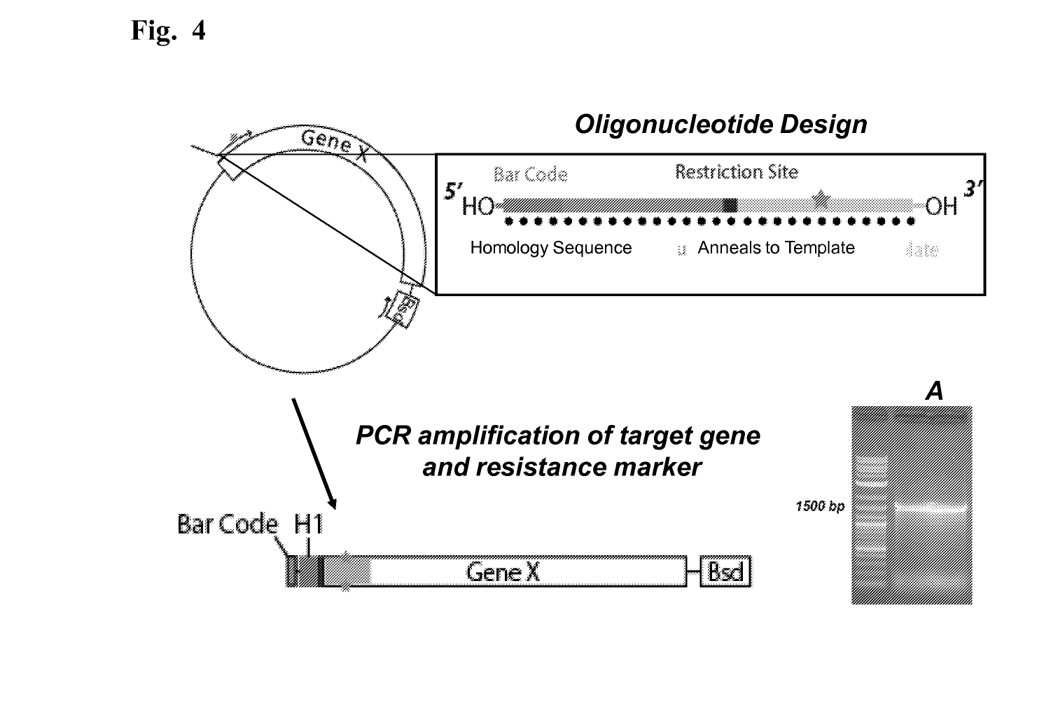

FIG. 4 (see also FIG. 2(2)) represents a sample oligonucleotide. Multiple oligonucleotides can be designed and synthesized in parallel such that the following features are present (5′-3′): a molecular barcode, homology region H1, a unique restriction site and a sequence annealing to the template containing a specific point mutation. Using the plasmid DNA from (1), ssDNA oligonucleotides are employed as primers in PCR reactions with a common downstream primer to amplify dsDNA containing a specific barcoded mutation (see the representative photo of an electrophoresis gel lane A, DNA at ˜1500 bp from PCR reaction with forward primer designed as described above).

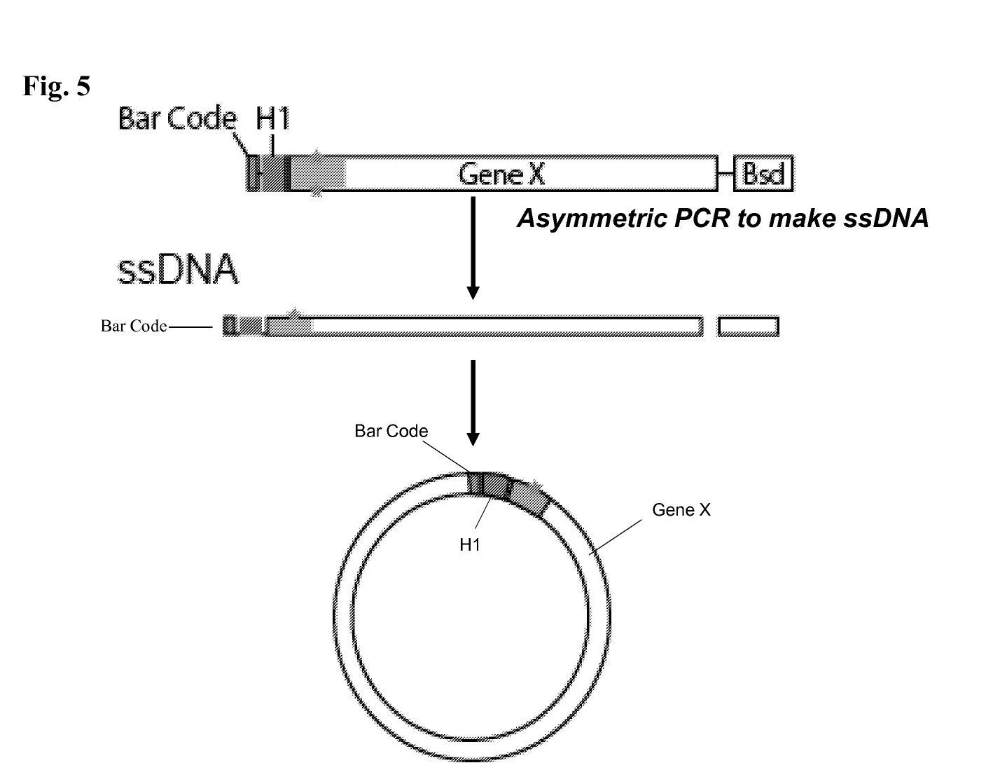

FIG. 5 (see also FIG. 2(3-4)). Amplified dsDNA is then used as a template in an asymmetric PCR reaction using for example a 1:1000 ratio of forward to reverse primer. Reverse primer is phosphorylated for subsequent circularization using CircLigase from Epicentre. In principle, the asymmetric PCR reaction is optional as a linear dsDNA molecule can be circularized using T4 DNA ligase. However, according to the manufacturer, circularization of ssDNA using CircLigase yields little to none of the concatamers that can potentially form when circularizing dsDNA. In this example, formation of circular concatamers during circularization can result in the attachment of a barcode to mutations other than the intended mutation.

### Example 2

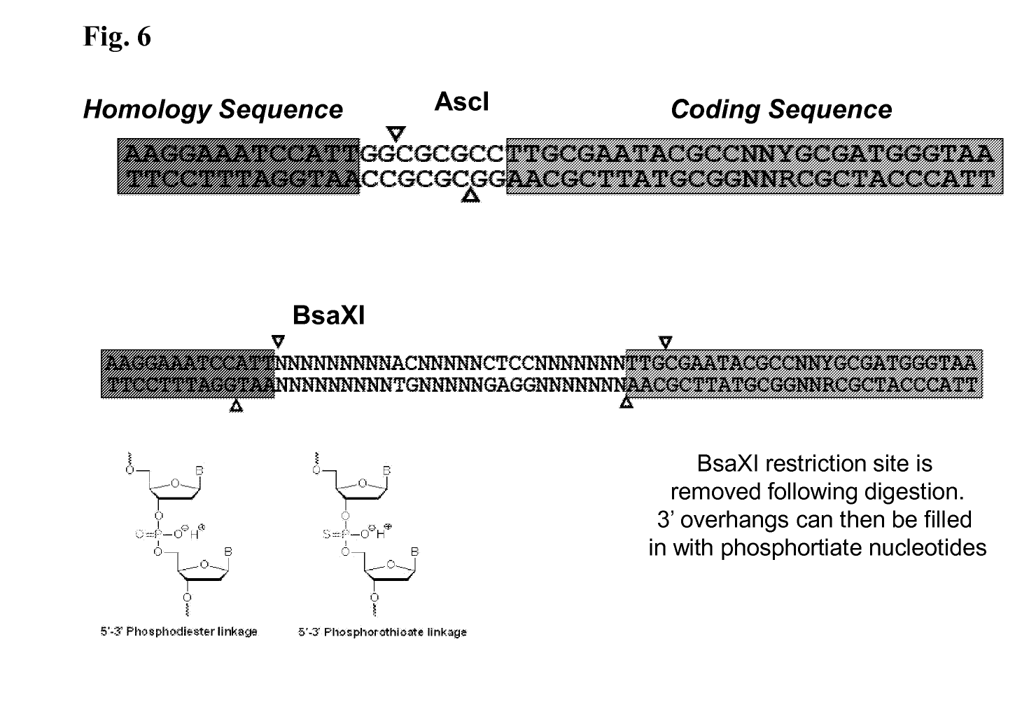

FIG. 6. Because the 5′ homology region in the final dsDNA cassette will interact with the coding region of the target mutated gene, it is important to develop the strategy such that the only mutations present are the mutations designed. Typical restriction sites can generate DNA overhangs containing DNA mismatches in the homology region that can potentially introduce unwanted mutations via recombination (e.g. AscI restriction site). To circumvent this issue, a type IIG restriction enzyme (e.g. BsaXI) should be used. Type IIG restriction enzymes recognize discontinuous sequences and cleave on both sides of the recognition sites. Put another way, the Type IIG restriction site serves its purpose as a recognition site for the restriction enzyme and is subsequently removed from the DNA construct following digestion. In this example, the Type IIG restriction enzyme, BsaXI is used. The use of BsaXI provides the added benefit of generating 3′ DNA overhangs. These 3′ overhangs can be filled for example, using alpha-phosphorthioate dNTPs and DNA polymerase I (Klenow) large fragment. Previous work has demonstrated that recombination efficiency can be significantly improved via the incorporation of phosphorthioate-containing DNA.

### Example 3

In certain embodiments, a single-stranded (ssDNA) construct that can be used for both barcoded TRMR type mapping and recursive MAGE-like recombineering. (ssDNA can be readily synthesized using any method known in the art. For example, synthesis can be more efficient at recombineering than for dsDNA, and only require the lambda bet protein). A set of ssDNA constructs with the following design can be synthesized. From 5′ to 3′, each oligo will contain one 18-bp priming site (P1), a 40-nt targeting region, aconserved 18-nt region for the amplification of barcode tags (P3), a unique molecular barcode (10 nt), the T7 phage promoter (23 nt), a uniform 18-nt untranslated region (UTR), one of four ribosome binding sites designed to give rise to translation initiation rates of varying levels (0-6 nt), an 8-nt spacer, a second 40-nt targeting region, and a second priming site (P2, 18 nt). The total length of this construct would not exceed 200 nt, the current limit of one methodology, Agilent technologies, that parallel DNA synthesis.

These cassettes will enable the manipulation of expression at both the transcriptional and translational level of each gene in E. coli. Additionally, the incorporation of unique molecular barcodes for each construct facilitates the rapid mapping of phenotypes to genotypes—sequencing of a minimal 10-nt region provides an advantage of the short read pyrosequencing (faster, less expensive) and represents a 10-100 fold reduction in sequencing needs (e.g. 10-nt vs. 1000-nt for a full gene). The outside priming sites (P1 and P2) allow for the amplification of the individual ssDNA libraries out of a mixed pool of library designs. Recombination can be carried out in the E. coli chassis strain with an inducible T7 RNA polymerase gene integrated onto the chromosome. In principle, however, any phage polymerase and its orthogonal promoter could be used. Enzymatic assays will be used to validate this design (e.g. lacZ, gusA). Cassettes harboring the T7 promoter and each of the RBS variants can be integrated upstream of the lacZ and gusA genes located at different positions on the chromosome in E. coli. Standard enzymatic assays can then be used to confirm a range of expression levels at differing levels of T7 RNA polymerase induction.

### Next Generation Multiplex Mutational Strategies

These strategies will provide a foundation design that allows for easy expansion to a broader range of mutations than just changing downstream expression. Here, the mutational strategies can be expanded to include at a minimum alteration of regions affecting protein activity, regulation of protein activity, and regulatory regions that perturb regulatory networks.

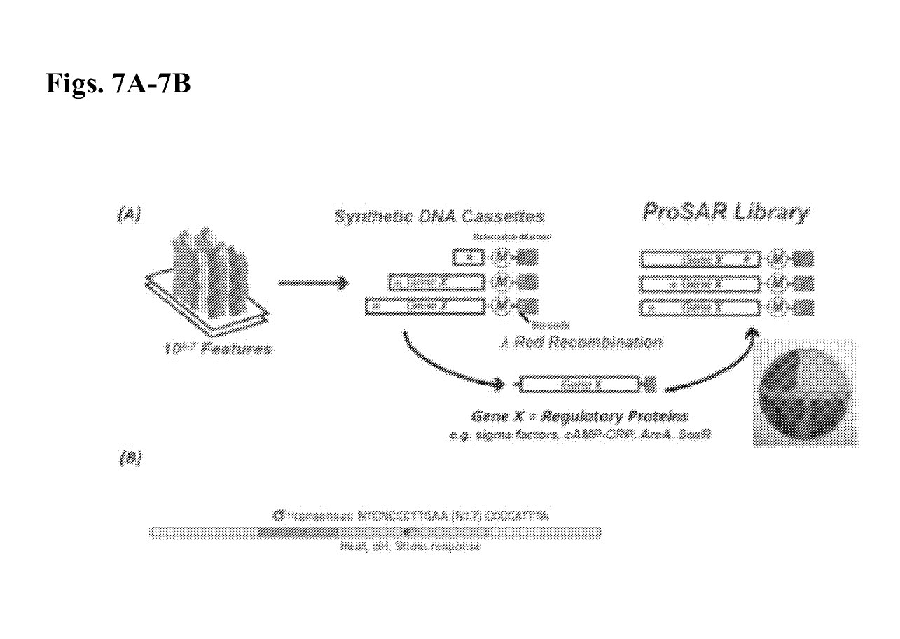

In preliminary studies, an approach has been designed and validated for using multiplex recombineering to generate barcoded protein sequence to activity mapping libraries (see FIG. 7). Point mutations were inserted within the ORF at any/all positions that are linked to a barcode inserted outside of the ORF. This allows specific manipulation over a distance much greater than that which is accessible via current ssDNA oligo synthesis technology (<200 NT) and the sequencing of much shorter regions (e.g. a 10-20 NT barcode or in certain embodiments up to a 1,000 nt barcode depending on the gene or gene segment) that are accessible by the highest throughput and lowest cost pyrosequencing approaches. Here, methods can be built upon this basic cloning strategy to develop whole new approaches for mapping mutations onto a cellular-level phenotype of interest (such as tolerance to isobutanol for mass production etc or biofuels production).

FIG. 7 represents a Multiplex Recombineering based Protein Sequence to Activity Relationship Mapping Concept. A) Beginning with custom synthesized oligonucleotides, dsDNA cassettes can be created such that each contains a single point mutation, a selectable marker and a unique barcode. With recombination, each of these cassettes can be integrated into genes encoding for any protein of interest (e.g. sigma factors, cAMP-CRP, ArcA, SoxR, etc.) ultimately yielding a barcoded library of designed point mutations or insertions of various sizes. In preliminary data, all aspects of this strategy have been validated, as illustrated in the inset figure via the introduction of an amber mutation within the galK gene of E. coli. B). An example of a ssDNA cassette to be used for recombineering. Here, this cassette will integrate the sigma32 consensus sequence into the promoter of its targeted gene in a barcoded fashion thus “rewiring” the sigma32 regulatory network in a trackable manner. In principle, any regulatory element (e.g. operators) can be introduced upstream of any gene with this design.

While it is expected to generate a range of approaches in this task, initial efforts focused on the following few library designs: i) Multiplex Recombineering driven Transcriptional Machinery Engineering: Barcoded libraries can be created of regulatory proteins that act on regulons of various sizes (e.g. σ factors, cAMP-CRP, ArcA, SoxR) containing complete alanine maps of all residues as well as complete substitution maps of all of the amino-acids forming the DNA binding/recognition region. Residues affecting regulator binding can be identified and thus perturb regulatory network activity in a manner that improves production. ii) Efflux pump engineering. Barcoded libraries can be generated of efflux pumps in E. coli that include complete alanine maps of all residues as well as complete substitution maps for all amino-acids thought to line or influence the pump core. Residue modifications that improve activity on isobutanol will be identified, thus enhancing tolerance and overall production will be identified; iii) Redox engineering. Barcoded libraries of enzymes involved in NADH/NAD(P)H metabolism where amino-acid sequences expected to interact with NADH or NAD(P)H will be substituted to allow for switching between the two co-factors. Modifications in enzymes that affect the larger NADH/NAD(P)H redox network, and potentially improve production of ethylene/isobutanol via improved co-factor availability can be found and constructed. Strategies to map genes onto traits of interest by connecting individual genes into larger regulatory or metabolic networks can be generated. This can be accomplished by grafting, in a barcoded manner, regulatory recognition sequences (or promoters) into the promoter region or ORF of a gene (e.g reverse gTME). As above, several proof-of-principle approaches can be performed i) replace the promoter regions of all genes with a barcoded glnAp2 promoter, which is known to respond to cellular glucose availability, ii) create barcoded libraries that integrate the soxS operator upstream of every gene in the genome, which will allow us to map one gene at a time expansions of the SoxR/S regulon onto traits of interest, and iii) expand to several other regulatory networks (such as σ32, cAMP-CRP, ArcA) as benefits the overall goals of the project.

### Next-Gen TRMR-MAGE Library Selection and Screening for Ethylene/Isobutanol Production

Here, tools to identify a broad range of genetic strategies for improving production of target compounds can be generated. This data will then permit development of a new prototype chassis strain. For each library design, the selections/screens will be initially identified. Samples at various timepoints will be taken, to analyze changes in library populations by barcode sequencing. Changes in barcode frequency equate to the overall fitness of the allele of interest in the selection/screen evaluated. In that index tagging (using short primers) is a well established method for multiplexing sequencing run. Each sample will be barcoded, which itself will contain barcodes indicative of specific mutations. Thus, by sequencing only a short piece of DNA, the population can be rapidly mapped for changes for all library designs across hundreds or samples simultaneously. This capability effectively moves the rate limiting step in the analysis to PCR (which can easily be multiplexed to 1000's/day). This throughput for mapping “rational” mutations at the genome-scale onto traits of interest goes far beyond what has been accomplished in the past.

### Example 4

Double-stranded PCR products from synthesized oligonucleotides can be constructed that can be used as substrates for multiplex recombineering. Each oligo will be designed to contain a unique barcode corresponding to the mutation it carries, which permits rapid sequence-activity mapping all designed mutations in parallel. Then, the ability to create comprehensive ProSAR libraries in parallel directly from single-stranded oligo pools will be generated. As sequencing technology advances, the need for barcodes that link to given mutations decreases. PRO-SAR libraries using ssDNA will be generated. The technology will be used to engineer several model proteins. The specific proteins of interest have applications ranging from therapeutic to pharmaceutical to biotechnological. At the completion of this project, novel, improved versions of each of the model proteins will be generated, thereby demonstrating the ability to gain understanding from the process and use that knowledge to engineer proteins.

Understanding the relationship between a protein's amino acid structure and function is critical in protein engineering efforts, which are increasingly commonplace in almost all drug development programs (e.g. whether focused on protein-based therapies or enzyme driven synthesis of pharmaceutical products). Now, protein design criteria grow increasingly stringent, including efforts to simultaneously alter multiple characteristics such as overall stability, catalytic activity, pharmacokinetic activity, shelf life, among others depending on the application.

Protein sequence-to-activity relationship (ProSAR) mapping is important in a broad range of basic, applied, and clinical efforts. For example, single missense mutations in the amino acid sequence of proteins have been implicated in many genetic diseases (e.g., sickle cell anemia, Golabi-Ito-Hall syndrome, Marfan's syndrome, and others). Often these mutations occur in the context of other SNPs and thus are difficult to characterize precisely. Also, spatial aggregation propensity mapping (SAP) has led to identification of mutations to confer greater stability in therapeutic antibodies. Finally, point insertion of fluorescent residues such as tryptophan permits researchers to study conformational changes to develop hypotheses on structure and ligand binding. Multiplex-ProSAR approach will enable such studies (and others) by allowing researchers to identify relevant mutations much more efficiently than is currently possible.

A method of quickly creating a range of mutations at single residues throughout a protein would have broad impact for protein science and engineering. Coupled with a sufficiently high-throughput screen or selection, important residues and mutations could be quickly identified and tested in a combinatorial manner to iteratively improve the desired protein function. Such a method would provide a more precise understanding of individual amino acid contributions, and in doing so provide a new strategy for directed exploration of protein sequence space.

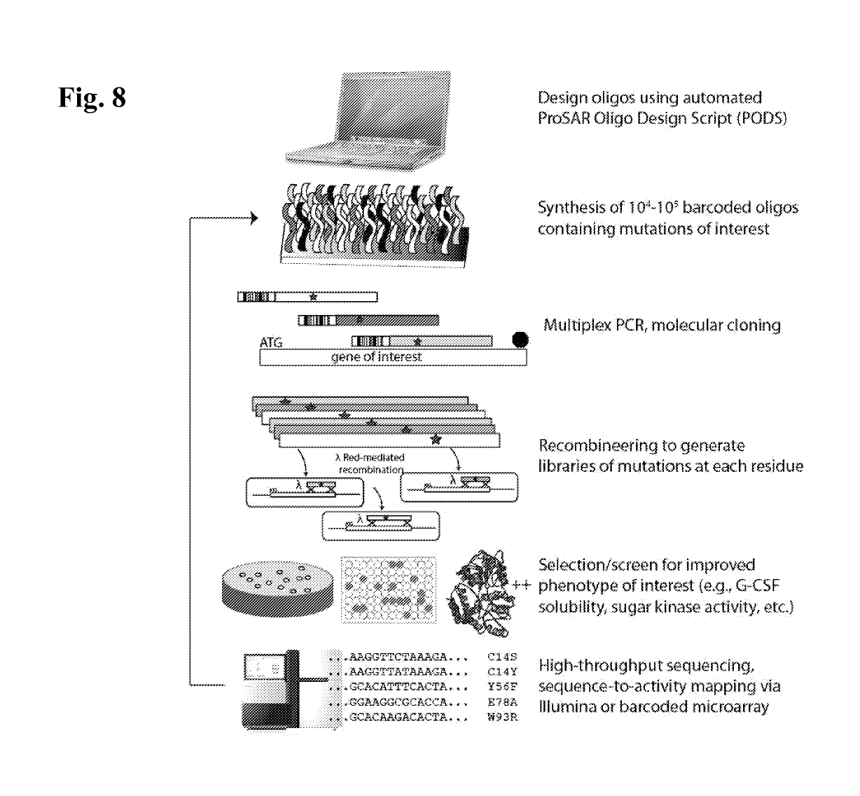

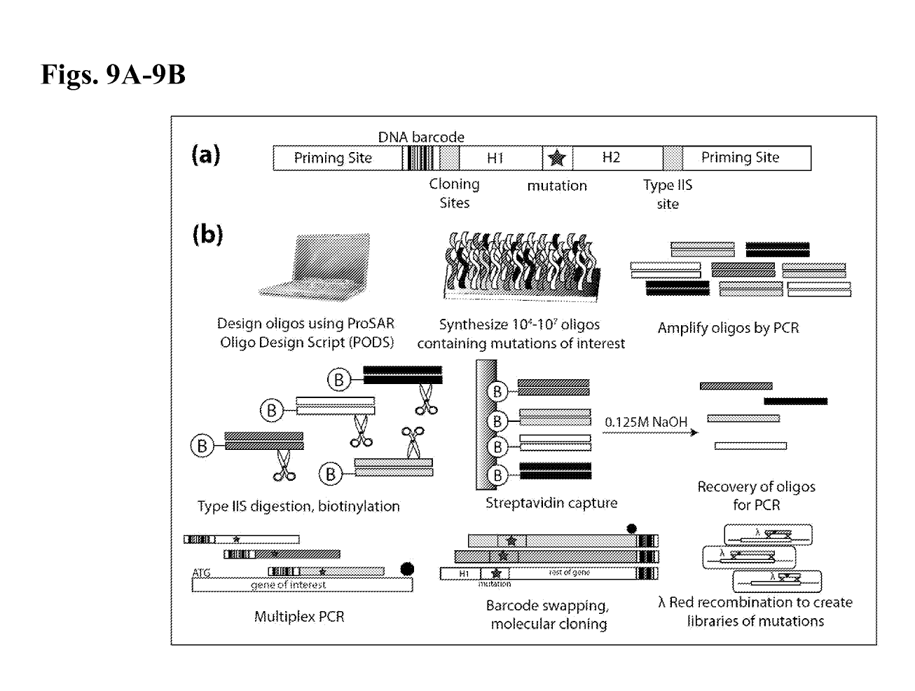

The technology described here uniquely combine these technologies to dramatically increase capabilities for (1) constructing rational protein libraries and (2) characterizing sequence-to-activity relationships by high-throughput screening and sequencing. FIG. 8 illustrates an exemplary strategy for multiplex recombineering ProSAR. FIGS. 9A and B illustrate (a) an exemplary design of the synthetic oligonucleotide and (b) an oligo amplification process from design to recovery. Recovered oligos will be used in the next steps of library creation.

This approach creatively combines multiplex oligonucleotide synthesis with recombineering (recombination-based genetic engineering), to generate custom-designed mutation libraries either within the genome or extra-chromosomally on a bacterial artificial chromosome (BAC) or plasmid of choice. Creation of directed libraries of amino acid substitutions at each residue on only one given protein is time- and resource-intensive using current methods. Conservatively estimating that ten individual residue libraries could be made in parallel by restriction/ligation, library construction for an average sized protein (ca. 200 amino acids) takes on the order of months. In comparison, the current approach allows for creation of multiple protein-wide libraries in a single week. The number of designed mutations is limited only by the number of synthetic oligos, tens of thousands of which can be synthesized on microarrays for a few thousand dollars and over a few weeks. Recovery of oligos from the microarray takes approximately one day (FIG. 9b). Using these oligos as primers, single-stranded multiplex PCR permits synthesis of all mutations at once. In this approach, recombineering replaces traditional molecular cloning, allowing construction of mutation libraries in parallel. The incorporation of a barcode corresponding to a given mutation (FIG. 9a) greatly streamlines analysis of both naïve libraries and clones selected for better performance as high-throughput sequencing generates millions of reads of short (ca. 100 bp) DNA sequences. Create comprehensive, barcoded ProSAR mapping libraries from oligonucleotides

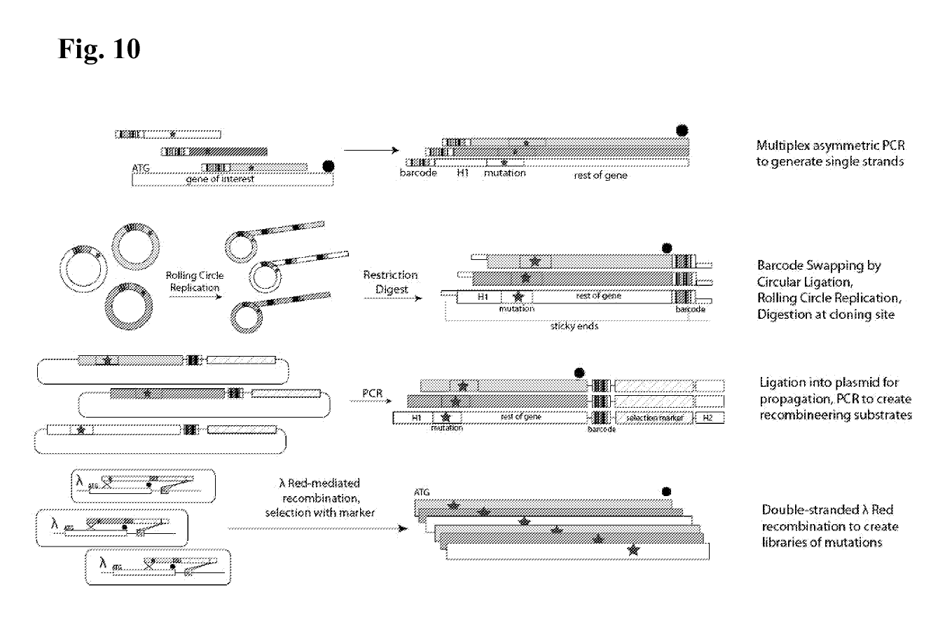

Libraries of barcoded mutations in individual residues using custom-synthesized oligonucleotide arrays can be created. FIG. 9 provides an overview of an exemplary version of the process. Briefly, modular oligos containing DNA barcodes, homology to the gene encoding the protein of interest, and a desired mutation are synthesized in multiplex on a oligonucleotide microarray. Oligos are recovered from the array to be used in asymmetric multiplex PCR, creating barcoded ssDNA libraries. The barcode is then moved outside the ORF of the gene of interest by circularization and digestion (FIG. 10 provides a schematic of a barcode swapping process). The resulting product becomes the substrate for double-stranded multiplex recombineering, creating libraries of mutations on the gene of interest in parallel.

Oligo Synthesis, Amplification, and Recovery. Oligonucleotide arrays containing up to 120,000 individual oligos are commercially available from Agilent. Previously, this technology was used to generate approximately 11,000 custom-designed 180-mers. Creating the thousands of oligos necessary for each protein of interest requires automation of the oligo design process. To this end, a simple computer program was created which, given an input of a gene and approximately 40 bp of genomic context, will rapidly design oligos of interest and assign the corresponding barcodes. In one example, because Agilent oligonucleotide arrays contain 10 pmol of total DNA, PCR amplification is necessary prior to use in subsequent cloning steps. This amplification protocol is similar to that employed previously, where novel priming sites for the gene of interest were created for selective amplification out of a mixed oligo pool. PCR results in double-stranded 120-mers, which will then be digested to remove the priming sites and create a 5′ overhang just before the barcode, which is subsequently filled in by biotinylated nucleotides. The biotinylated double strands can be captured on a streptavidin column then denatured with weak sodium hydroxide to recover the non-biotinylated ssDNA. The ssDNA 120 mers are then purified for use in construction of the barcoded mutation libraries.

### Generating Barcoded Mutation Libraries

The ssDNA 120 mers are used as the forward primers in an asymmetric PCR reaction (see FIG. 10). Because oligos anneal to the gene of interest at different locations along the gene (as defined by the intended mutation), the asymmetric PCR reaction creates single-stranded DNA of varying lengths, all of which contain the designed mutations and their respective barcodes on the coding strand. However, insertion of a barcode without disrupting the open reading frame requires that the barcode lie outside of the ORF of the gene of interest. Thus, prior to recombineering, the ssDNA fragment product of the asymmetric PCR is circularized using for example, CircLigase, a ligase specific to ssDNA. This step allows for rolling circle replication (RCR) of the circularized product. The product of RCR is a fragment comprising continuous, double-stranded repeats of the sequence of the circular ssDNA. The double-stranded product will then be digested with restriction enzymes, leaving a product where the barcode is located 3′ of the stop codon of the gene of interest. Once the barcode is moved downstream of the ORF, the library of products can optionally be cloned into a vector containing a selection marker of interest (a range of different markers can be used e.g., auxotrophy (URA3), resistance (KanR), etc.). From this vector, a final PCR reaction creates the dsDNA substrates for λ-Red mediated recombination into E. coli.

Detailed protocols for double-stranded recombination with λ-Red have been described. Briefly, transformation of linear dsDNA PCR products coupled with expression of the recombinase genes bet, gam, and exo from phage λ leads to very efficient incorporation of the product into the bacterial genome or into a extra-chromosomal vector by homologous recombination. In this case, the homology regions of interest are H1: inside the gene itself (prior to and after the mutation) and H2: downstream of the gene of interest. Using this method, an entire library of mutations corresponding to the custom-synthesized oligos will be transformed and recombined. Recombinants are selected by plating on appropriate media (e.g. lacking uracil, if URA3 marker is used).

FIG. 10 represents a schematic of steps in library construction between oligo recovery and double-stranded recombination. Oligos contain barcodes which map to the mutation of interest, but cannot be present in the ORF of the gene of interest. After PCR amplification, barcode swapping relegates the barcode to the 3′ region.

### Example 5

In one exemplary method, galactokinase (GalK) is used as a model protein for methods described herein for developing and optimizing protocols. GalK was chosen because it is located in the E. coli genome, as well as on existing plasmids in our lab, based on experience in a variety of screens and selections for sugar kinase function, there is a crystal structure, and key residues have been mapped to function previously.

In these studies optimal mutation distances will be examined as well as examining mutations in cellular DNA repair mechanisms that are known to affect efficiency (e.g., MutS mismatch repair, DNA polymerase proofreading). In addition, an alternative to dsDNA recombineering is the use of ssDNA oligos directly as recombineering substrates.

This technology will be a broadly applicable technology for design, construction, and analysis of barcoded libraries of point mutations in many proteins of interest on a time scale orders of magnitude faster than current molecular cloning methods allow.

### Example 6

Create Comprehensive ProSAR Libraries in Parallel from Single-Stranded Oligo Pools

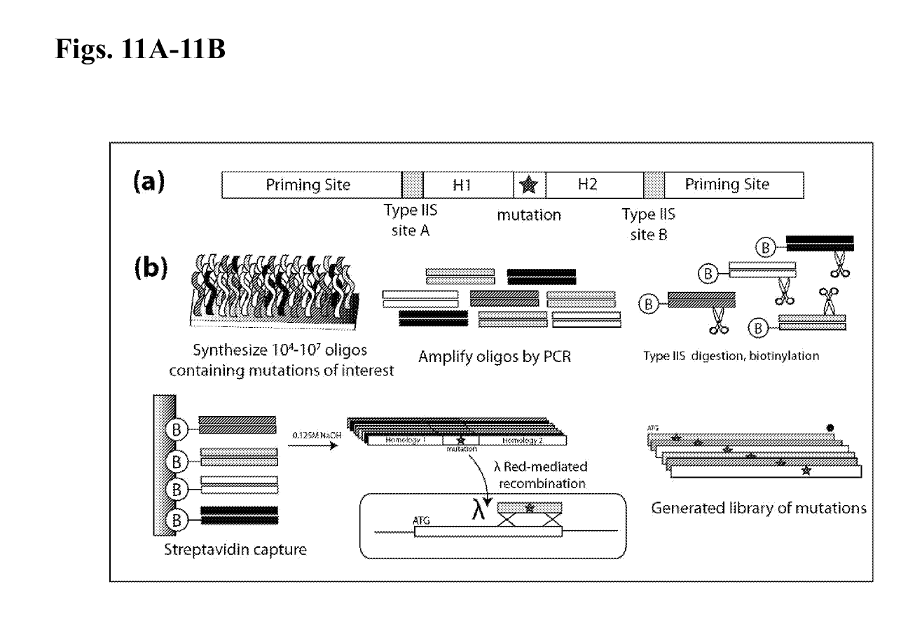

Libraries similar will be created by using ssDNA oligos directly as substrates for multiplex recombineering. A similar oligo design as detailed above permits recovery of mutation-containing oligos otherwise completely homologous to the gene of interest. Single-stranded Recombineering. Oligo-mediated allelic replacement (OMAR) uses single-stranded DNA oligos for recombineering. Oligos will be recovered from the synthesized array and transformed directly into cells expressing the λ Red recombinase genes (note that only bet is needed for ssDNA), thus creating point mutations in the targeted gene. One advantage of this approach is that it eliminates the molecular biology steps required to implement barcoding. FIG. 11 illustrates the entire process of library creation using ssDNA (compare to FIGS. 9 & 10). One current disadvantage of this approach is that barcodes can be used to encode more information in a shorter sequence of DNA, thus reducing sequencing requirements and allowing for use in massively parallel multiplexed sequencing machines that have shorter read lengths (roughly 100 bp). However, as sequencing technology advances, the need for barcodes that link to given mutations decreases. For example, the Pacific Biosciences RS system can generate millions of reads up to 1000 bp in length. A second consideration is that the barcoded dsDNA strategy provides a measure of confidence that each mutant contains only the single point mutation of interest, as opposed to the possibility of inserting multiple mutations via the more efficient ssDNA multiplex recombineering protocols (about 103-4 better).

The process for amplification and recovery of oligos from the array is nearly identical to that discussed previously. One difference is the placement of type IIS restriction sites on the 5′ and 3′ ends of the mutation (FIG. 11a). The purpose of this strategy is to cut away priming sites, restriction sites, etc. on both sides of the oligo, leaving a single-stranded DNA fragment that is entirely homologous to the genomic template except for the mutation of interest. Once purified, these oligos can serve as the substrates for λ Red recombination (E. coli strains already engineered for highly efficient ds- or ssDNA recombineering can be used). Single-stranded recombineering protocols have been employed often, most notably in the case of MAGE, where an automated process for iterative creation of point mutations in the genome was developed. In addition, after recombineering, phenotypic selection, and sequencing the process to build up combinatorial libraries for multi-trait protein optimization can be used.

FIGS. 11A-B represent a schematic of library construction using single-stranded oligonucleotides. (a) General oligo design (ex. FIG. 9b) (b) Oligo recovery and recombineering for library generation.

One creative aspect of this strategy is that oligos can be designed (compare FIGS. 9a and 11a) such that the same oligonucleotide array can be used to generate both double-stranded and single-stranded substrates for creation of mutation libraries (thus providing some flexibility for broader use). Digestion with a type IIS restriction enzyme (such as BsaI which leaves a 5′ overhang between the barcode and the homology sequence allows for selective biotinylation and capture of oligo sequences that are recombination ready.

One trade-off between the dsDNA and ssDNA strategies is the relative confidence that created libraries contain only the targeted mutations. For example, ssDNA recombination can be much more efficient than dsDNA, thus raising the possibility that individual library members may contain more than one point mutation when created by the ssDNA approach. A further consideration is that double-stranded, barcoded libraries allow for a selection for recombinants via a resistance or auxotrophy marker, thus minimizing the presence of individual library members that have no mutation at all. Thus, while the cloning steps are simplified, which should aid in the dissemination of our approach by the broader community, there are some limitations w/respect to confidently mapping sequence-to-activity relative to the dsDNA approach.

### Sequence-to-Activity Mapping

Once libraries are created, they will be subjected to screens or selections for the phenotype of interest. The best performers from these screening protocols will be recovered and analyzed by sequencing. The short (10-25 bp) DNA barcode that is linked to a given mutation allows determination of individual mutations that produce the given phenotype without necessitating sequencing of the entire gene. High-throughput sequencing technology is capable of millions of runs on short DNA sequences (ca. 100-200 bp), which generates enough data to completely analyze an entire library of barcodes in one sequencing cycle. Certain residues and mutations will be determined from sequencing readout that have varied responses (e.g. improved activity, stability etc.).

Once mutations are discovered and selected for a trait of interest, the method will be iterated to combinatorially engineer the phenotype of interest. Alternatively, these mutations will also be tested combinatorially by creating libraries using diversity-generating methods such as DNA shuffling. In this case, the presence of the barcode precludes the need for subsequent large-scale oligo synthesis since primers specific to a DNA barcode can amplify relevant mutations from the same oligo array.

In certain exemplary methods, model proteins in this study can have applications for pharmaceutical synthesis, metabolic engineering, protein-small molecule interactions, and therapeutic protein production. An overview of the proteins is given in Table 1.

TABLE 1
Proteins to be engineered in this study. Unless otherwise
indicated, proteins are from E. coli.

| | | | |
|---|---|---|---|
| Galactokinase | High-throughput | 20 AA satu- | Phosphorylation |
| (GalK) | colorimetric | ration at five | of novel sugars |
| | screen | catalytic | for drug |
| | | residues | glycosylation |
| Dihydrofolate | trimethoprim | alanine | Antibiotic |
| reductase (FolA) | res. | scan | resistance |
| Homoserine O- | growth at 42C, | 20 AA | Metabolic |
| succinyltransferase | growth on | saturation | engineering, |
| Human Granulocyte | Protein folding | 20 AA | Therapeutic |
| Colony Stimulating | reporter | saturation | protein |
| | | | production |
| Human G-protein | Protein folding | 20 AA | Drug/receptor |
| coupled receptors | reporter | saturation | interactions, |
| 5HTR1A and | | | protein |
| CRFR1 | | | structure |
| | | | determination |

In certain exemplary methods a complete substitution library of a pharmaceutically protein (e.g. GCSF), not produced at high levels can be produced using recombinant strategies (e.g. expression in a microbial host). Then, the library can be screened to using this barcoding strategy substitutions for improving expression of the target protein in soluble form. New libraries containing combinations of substitutions that improve expression can be created and perform additional screening/selections can be performed to identify superior combinations. In other methods, proteins that are difficult to get crystal structures for can be pursued as above. In addition, heterologous proteins required for introducing novel metabolic pathways into microbes of interest can be pursued by these methods.

All proteins are model proteins for which a high-throughput assay exists for the phenotype of interest. In the cases of G-CSF and the G-PCR proteins, one object of these engineering efforts will be increasing the overall folding and solubility of these proteins. Point mutations in these proteins have been found to convey significant changes in solubility in the context of their respective protein folding reporters. The wide variety of proteins was chosen to showcase proteins with different health-related applications and to provide evidence of proof-of-principle for this methodology. These methods will make every intended mutation in parallel and (2) sequence analysis of the libraries will be faster in high-throughput format.

Screens will be designed such that the wild type activity will be a baseline for comparison of phenotypes. For example, when judging trimethoprim resistance as in the case of FolA, the minimum inhibitory concentration will be that of the wild-type FolA. Multiple colorimetric screens, antibiotic or auxotrophic selections exist, and periplasmic protein folding/solubility reporters including those in Table 1, and in the use and design of both positive and negative controls will be used.

After library construction, analysis, and iteration, a range of GalK mutations that broaden sugar specificity, FolA mutations that affect trimethoprim binding, MetA residue changes that increased thermostability, and G-CSF and G-PCR mutations that increase overall expression in E. coli will be discovered.

Preliminary data will demonstrate that the method works to (1) amplify oligonucleotides and recover the correct strands, (2) perform PCR in multiplex, and (3) incorporate barcodes in the proper configuration via circularization.

To simulate the dilute nature of the oligo mixture when recovered from the array, a small amount (ca. 0.1 pmol) of each of five degenerate oligos encoding mutations at five different residues of E. coli galactokinase (GalK) was mixed for amplification by PCR. The product was then digested with NdeI and the overhang filled in with Klenow polymerase and biotinylated UTP. Capture on streptavidin beads and denaturation with 0.125 M NaOH led to release of single stranded oligos. The oligos were purified using Qiagen Nucleotide Removal Kit.

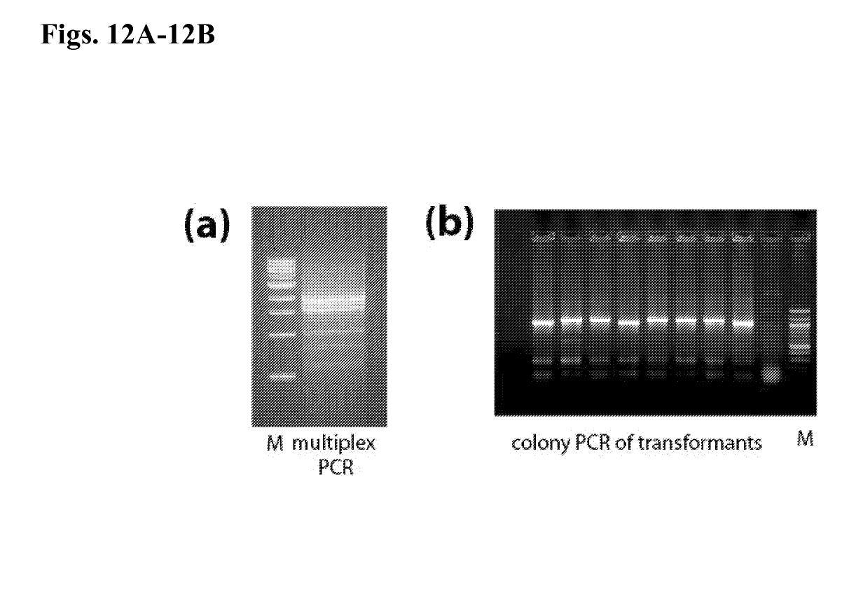

Next, asymmetric PCR (as in FIG. 10) was performed using the GalK gene as a template and the recovered oligos as the forward primers. As expected, the PCR reaction generated five bands of different lengths (because of the location of each mutation) (FIG. 12a). These bands were then gel extracted and subjected to circular ligation with CircLigase (Epicentre). After circular ligation was complete, the circular DNA was digested with EagI and AgeI, the “cloning site” enzymes from FIG. 9a. Again, this digestion led to five distinct bands, each corresponding to a double-stranded product of a different length. Colony PCR on a small sampling of clones revealed at least two different bands (FIG. 12b) and sequencing of these bands confirmed the location of the barcode outside the ORF of interest on the 3′ end.

FIGS. 12A-12B represent (a) Assymetric PCR with five oligos in multiplex; (b) Colony PCR on a small sample of transformants after barcode-swapping.

The foregoing discussion of the invention has been presented for purposes of illustration and description. The foregoing is not intended to limit the invention to the form or forms disclosed herein. Although the description of the invention has included description of one or more embodiments and certain variations and modifications, other variations and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A construct comprising, a gene or gene segment capable of encoding a target protein, the gene or gene segments having a traceable barcode positioned outside of the gene or gene segments open reading frame wherein the traceable barcode corresponds to or is quantitatively linked to a genetic variation of the gene or gene segment.

2. The construct of claim 1, wherein the genetic variation comprises a point mutation.

3. The construct of claim 1, wherein the traceable barcode comprises a nucleic acid sequence.

4. The construct of claim 1, wherein the constructs can be compiled together to make a library of one or more target proteins or a trait.

5. The construct of claim 1, wherein the genetic variations together in a pool represent every mutated residue of the target protein.

6. The construct of claim 1, wherein the target protein is a prokaryotic protein.

7. The construct of claim 1, wherein the target protein is a eukaryotic protein.

8. The construct of claim 4, wherein the pool comprises every mutated residue for all genes of a genome capable of encoding a protein.

9. The construct of claim 1, further comprises a selected construct for optimum function of the target protein.

10. The construct of claim 1, wherein the construct is an optimized target protein of a pathway.

11. A method for generating a construct comprising:
obtaining one or more oligonucleotide sequences, each containing barcode sequences, regions of homology to one or more target gene(s), and regions of genetic variation towards one or more target gene(s);
using the one or more oligonucleotide sequences to generate amplified constructs comprising regions of homology suitable for homologous recombination;
circularizing the amplified constructs; and
digesting the circularized constructs to form constructs comprising barcodes outside of open reading frames (cassettes) of the one or more targeted gene(s).

12. The method of claim 11, further comprising, recombining the constructs to form a library of barcoded mutant target genes.

13. The method of claim 11, further comprising using the cassettes for recombineering or parallel tracking of more than one target protein.

14. The method of claim 12, wherein constructs for more than one target protein in a pathway are generated.

15. The method of claim 12, wherein multiple genetic variations are introduced to generate constructs covering every possible naturally-occurring and non-natural amino acid residue of the target protein.

16. The method of claim 11, wherein the restriction site is a Type IIG restriction site.

17. A method for generating an in vivo construct library comprising generating constructs of claim 1 wherein each construct represents one genetic variation in a target gene of a target protein and the construct library comprises all naturally-occurring and non-natural amino acid residue changes of the target protein.

18. A method comprising:
assigning ranks pertaining to biological effects of genetic variations of a plurality of genes or genetic loci capable of coding for a target protein;
assigning ranks pertaining to the biological effect due to the genetic variations of the plurality of genes or genetic loci;
obtaining and analyzing one or more rank(s) of the genetic variations of the genes or genetic loci pertaining to a predetermined selection process;
obtaining one or more composite rank(s) based on the ranks of the biological effects as they pertain to the predetermined selection process and biological context rank; and
designing a genomically-engineered process, cell or organism based on the composite rank(s).

19. The method of claim 18, where a biological effect comprises modulating the target gene.

20. The method of claim 19, wherein the target gene comprises an enzyme and modulating the target gene comprises increasing biological activity of the enzyme compared to a target gene not having the genetic variation.

21. The method of claim 18, where the assigning comprises measuring the effect of the genetic variation on a specific trait.

22. A computer-readable medium having computer-readable instructions, which, when executed by a computer, cause the computer to carry out a method comprising:
receiving first gene(s) or genetic segment score representing a score of a biological effect or condition due to a genetic variation of a gene or gene segment of a target protein;
receiving at least a second gene(s) or genetic score representing a second score of another genetic variation of the target protein;
combining the scores; and
assigning a combined score related to one or more genetic variations in order to assess a value of the genetic variations related to a trait for the target protein.

23. The computer-readable medium of claim 22, further comprising designing a genomically-engineered organism or cell based on the composite scores for two or more genes or genetic loci.

24. The computer-readable medium of claim 22, wherein information related to more than one target gene can be received and assessed.

25. A system comprising:
a component for assessing a score of a genetic variation of genes or genetic segments pertaining to a trait of one or more target proteins; and
a component for reporting the score of the genetic variation of genes or genetic segments pertaining to a trait of one or more target proteins; and
a component for compiling the scores of one or more target proteins.

26. The system of claim 25, wherein the genetic variation comprises a mutation, insertion, deletion or other genetic variation.

27. A library comprising constructs of claim 1.

28. The library of claim 27, wherein the library is a genomic library of a target microorganism.

29. The library of claim 27, wherein the constructs comprise all possible genetic variations together in a pool representing every mutated residue of the target protein.
